(19) United States
(12) Patent Application Publication
(10) Pub. No.: US 2025/0038269 A1
(43) Pub. Date: Jan. 30, 2025

(54) BATTERY ELECTROLYTE SOLUTION AND BATTERY

(71) Applicant: ZHUHAI COSMX BATTERY CO., LTD., Zhuhai (CN)

(72) Inventor: Yingdi MU, Zhuhai (CN)

(73) Assignee: ZHUHAI COSMX BATTERY CO., LTD., Zhuhai (CN)

(21) Appl. No.: 18/914,024

(22) Filed: Oct. 11, 2024

Related U.S. Application Data
(63) Continuation of application No. PCT/CN2023/104851, filed on Jun. 30, 2023.

(30) Foreign Application Priority Data
Aug. 25, 2022 (CN) 202211025041.7

(51) Int. Cl. H01M 10/0567; C07D 327/10; H01M 10/0525; H01M 10/0568; H01M 10/0569

(52) U.S. Cl. CPC H01M 10/0567; C07D 327/10; H01M 10/0525; H01M 10/0568; H01M 10/0569

(57) ABSTRACT

Disclosed are a battery electrolyte solution and a battery. The battery electrolyte solution includes an organic solvent, an additive, and an electrolyte salt, and the electrolyte solution is in contact with a negative electrode plate; the organic solvent includes an ethyl group solvent, the additive includes fluoroethylene carbonate, the electrolyte salt includes a lithium salt, and percentages of the ethyl group solvent, the fluoroethylene carbonate, and the lithium salt in a total mass of the electrolyte solution are configured as follows: 0.4−N³≤A+B²+C²≤5.2−N³, where N denotes a peeling strength value of the negative electrode plate, A, B, and C respectively denote a percentage of the mass of the ethyl group solvent, the fluoroethylene carbonate and the lithium salt in the total mass of the electrolyte solution.

### CROSS-REFERENCE TO RELATED APPLICATIONS

The present disclosure is a continuation of International Application No. PCT/CN2023/104851, filed on Jun. 30, 2023, which claims priority to Chinese Patent Application No. 202211025041.7, filed on Aug. 25, 2022. All of the aforementioned patent applications are hereby incorporated by reference in their entireties.

### TECHNICAL FIELD

Embodiments of the present disclosure relate to the field of lithium-ion battery technologies, and in particular, to a battery electrolyte solution and a battery.

### BACKGROUND

A structure of a positive electrode active material in a lithium-ion battery is unstable at a high temperature, and metal ions are easily dissolved and deposited on a surface of a negative electrode plate. This destroys a structure of a solid electrolyte interface (Solid Electrolyte Interface, SEI) film on the surface of the negative electrode plate, which causes a continuous increase in a negative electrode impedance, and further causes a continuous increase in a temperature of the battery, and causes a safety accident when heat accumulates and cannot be released.

Currently, a flame retardant is generally added to an electrolyte solution to improve safety performance of a battery at a high temperature, but adding the flame retardant may lead to degradation of other performance of the battery than safety performance. Therefore, how to improve safety performance of a battery while avoiding degradation of other performance of the battery has become an urgent problem to be resolved.

### SUMMARY

Embodiments of the present disclosure provide a battery electrolyte solution, a preparation method of the battery electrolyte solution, and a battery, which resolves a problem of how to improve safety performance of a battery while avoiding degradation of other performance of the battery.

To achieve the foregoing purpose, according to a first aspect, an embodiment of the present disclosure provides a battery electrolyte solution. The battery electrolyte solution includes an organic solvent, an additive, and an electrolyte salt. The organic solvent includes an ethyl group solvent, the additive includes fluoroethylene carbonate, and the electrolyte salt includes a lithium salt. The electrolyte solution is in contact with a negative electrode plate. Percentages of the ethyl group solvent, the fluoroethylene carbonate, and the lithium salt in a total mass of the electrolyte solution are configured as follows:

0.4−N³≤A+B²+C²≤5.2−N³,

where N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

Optionally, the value of the peeling strength N of the negative electrode plate ranges from 0.1 gf/mm to 2 gf/mm.

Optionally, the mass of the ethyl group solvent accounts for 40 wt % to 85 wt % of the total mass of the electrolyte solution.

Optionally, the mass of the fluoroethylene carbonate accounts for 5 wt % to 18 wt % of the total mass of the electrolyte solution.

Optionally, the mass of the lithium salt accounts for 12 wt % to 18 wt % of the total mass of the electrolyte solution.

Optionally, the lithium salt includes at least one of lithium bis(trifluoromethanesulphonyl)imide, lithium bis(fluorosulfonyl)imide, or lithium hexafluorophosphate.

Optionally, the electrolyte solution further includes a thiophene compound, and a structural formula of the thiophene compound is as follows:

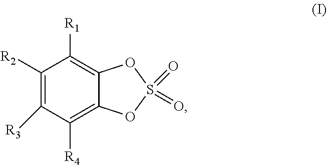

where R₁ is any one of hydrogen, halogen, and an alkyl carbon chain, R₂ is any one of hydrogen, halogen, and an alkyl carbon chain, R₃ is any one of hydrogen, halogen, and an alkyl carbon chain, R₄ is any one of hydrogen, halogen, and an alkyl carbon chain, and a quantity of carbon atoms in the alkyl carbon chain ranges from 1 to 10.

Optionally, the halogen is any one of fluorine, chlorine, and bromine.

Optionally, at least one carbon or hydrogen in the alkyl carbon chain is substituted by oxygen or halogen.

Optionally, a structural formula of the thiophene compound is any one of following:

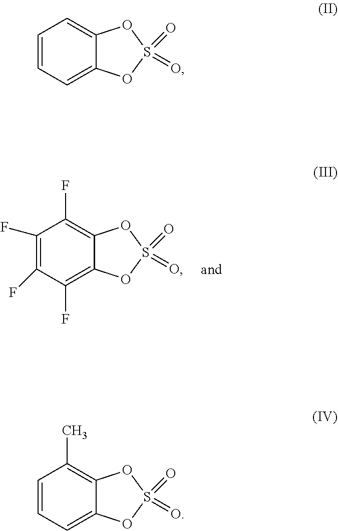

Optionally, a mass of the thiophene compound accounts for 0.1 wt % to 2 wt % of the total mass of the electrolyte solution.

According to a second aspect, an embodiment of the present disclosure provides a preparation method of a battery electrolyte solution. The method is used for preparing the battery electrolyte solution according to the first aspect. A percentage of each of the ethyl group solvent, the fluoroethylene carbonate, and the lithium salt in the battery electrolyte solution in the total mass of the electrolyte solution is determined based on a peeling strength expected to be reached after the negative electrode plate is infiltrated with the electrolyte solution, such that the percentages of the ethyl group solvent, the fluoroethylene carbonate, and the lithium salt in the total mass of the electrolyte solution meet:

0.4−N³≤A+B²+C²≤5.2−N³,

where N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

According to a third aspect, an embodiment of the present disclosure provides a battery. The battery includes a positive electrode plate, a negative electrode plate, and the battery electrolyte solution according to the first aspect. Both the positive electrode plate and the negative electrode plate are infiltrated with the battery electrolyte solution, and the battery meets the following expression:

0.4−N³≤A+B²+C²≤5.2−N³,

where N denotes a peeling strength of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

In embodiments of the present disclosure, the battery electrolyte solution is enabled to include an organic solvent, an additive, and an electrolyte salt. The organic solvent includes an ethyl group solvent, the additive includes fluoroethylene carbonate, and the electrolyte salt includes a lithium salt. Percentages of the ethyl group solvent, the fluoroethylene carbonate, and the lithium salt in a total mass of the electrolyte solution are configured as follows: 0.4−N³≤A+B²+C³≤5.2−N³. N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution. A relatively robust solid electrolyte interface (Solid Electrolyte Interface, SEI) film may be formed on a surface of the negative electrode plate while improving infiltration of the negative electrode plate. The SEI film covering the surface of the negative electrode plate inhibits a side reaction between a negative electrode active material and the electrolyte solution, so as to reduce accumulation of a side reaction product between the negative electrode active material and a current collector and on the surface of the negative electrode plate. In this way, a peeling strength of the negative electrode plate may be increased, which further reduces a negative electrode impedance (also reduces an internal resistance of the battery), thereby reducing a risk of self-heating and self-ignition of the battery, and improving safety performance of the battery.

In addition, the electrolyte solution is enabled to include the lithium salt that meets the foregoing relational expression, so that a sufficient lithium source may be provided for the battery, thereby increasing a lithium ion migration rate to improve conductivity, and providing a guarantee for low-temperature performance and long-cycling performance of the battery. In other words, the battery electrolyte solution provided in the embodiments of the present disclosure resolves a problem of how to improve safety performance of the battery while avoiding degradation of other performance of the battery.

### BRIEF DESCRIPTION OF THE DRAWINGS

To describe the technical solutions in the embodiments of the present disclosure more clearly, the accompanying drawing in this specification is described below. Apparently, the following accompanying drawing is merely an embodiment of the present disclosure, and a person of ordinary skill in the art may still derive other drawings from the listed accompanying drawing without creative efforts.

FIG. 1 is a schematic structural diagram of a battery according to an embodiment of the present disclosure.

### DETAILED DESCRIPTION OF THE EMBODIMENTS

The following clearly describes the technical solutions in embodiments of the present disclosure with reference to the accompanying drawings in embodiments of the present disclosure. Apparently, the described embodiments are merely some but not all of embodiments of the present disclosure. All other embodiments obtained by a person of ordinary skill in the art based on the embodiments of the present disclosure without creative efforts shall fall within the protection scope of the present disclosure.

An embodiment of the present disclosure provides a battery electrolyte solution. The battery electrolyte solution includes an organic solvent, an additive, and an electrolyte salt. The organic solvent includes an ethyl group solvent, the additive includes fluoroethylene carbonate, and the electrolyte salt includes a lithium salt. The electrolyte solution is in contact with a negative electrode plate. Percentages of the ethyl group solvent, the fluoroethylene carbonate, and the lithium salt in a total mass of the electrolyte solution are configured as follows:

0.4−N³≤A+B²+C²≤5.2−N³,

where N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

In the present disclosure, the battery electrolyte solution is a non-aqueous electrolyte solution.

It should be understood that N denotes a peeling strength value of the negative electrode plate, in a unit of gf/mm. The peeling strength value N may be set according to an actual situation, for example, N may be 0.5 gf/mm, 0.8 gf/mm, 1.8 gf/mm, 0.2 gf/mm, 0.3 gf/mm, 0.4 gf/mm, 1.6 gf/mm, or 0.9 gf/mm, or may be a range composed of any two of these values. A peeling strength of the negative electrode plate refers to a maximum force required to peel off a negative electrode active material layer per unit width, and is used to reflect a bonding strength between the negative electrode active material layer and a current collector. A method for testing a peeling force strength includes: cutting each negative electrode plate into a sample strip of 24 mm×15 cm, covering the strip with a glass slide, pressing the electrode plate back and forth by using a roller, and testing the strip with a tensile machine at a speed of 200 mm/min, to obtain a testing result that is a peel force of P (in a unit of gf). A calculation formula is as follows: Peeling strength N (gf/mm)=P/24 mm (width).

In a relational expression of the present disclosure, only a numerical portion of the peeling strength value rather than a unit portion participates in calculation. The foregoing relational expression is used as an example. For example, in Example 1 of the present disclosure, if the percentage A of the mass of the ethyl group solvent in the total mass of the electrolyte solution is 50% (0.5), the percentage B of the mass of the fluoroethylene carbonate in the total mass of the electrolyte solution is 10% (0.1), the percentage C of the mass of the lithium salt in the total mass of the electrolyte solution is 14% (0.14), and the peeling strength value is 0.8 gf/mm, N³+A+B²+C²=0.83+0.5+0.1²+0.14²=1.0416 (which is 1.04 after being rounded to two decimal places).

During specific implementation, a value of N³+A+B²+C² may be 0.45, 0.5, 0.6, 0.7, 0.8, 1.0, 1.5, 2.2, 3.0, 3.5, 4.0, 4.5, 4.78, 5.0, 5.2, or the like, or may be any value within a range composed of any two of these values. When the value of A+B²+C²+N³ ranges from 0.45 to 5.2 (including endpoints), a better synergistic effect is achieved between the negative electrode plate and the battery electrolyte solution. Specifically, when the value of A+B²+C²+N³ ranges from 0.45 to 5.2, surface tension of the electrolyte solution may be reduced, and the growth of lithium dendrites may be inhibited, so that a contact angle between the electrolyte solution and the negative electrode plate is reduced, thereby improving infiltration of the electrolyte solution, increasing a transmission rate of lithium ions, improving low-temperature discharge performance of the battery, improving interfacial compatibility of the battery, inhibiting a side reaction between the negative electrode plate and the electrolyte solution, which further reduces accumulation of a side reaction product between the negative electrode active material and the current collector and on the surface of the negative electrode plate, and also increases the peeling strength of the negative electrode plate, and reduces an internal resistance of the battery. In this way, safety accidents due to a continuous increase in a temperature of the battery may be avoided, and low-temperature performance of cells of the battery may be comprehensively improved.

In addition, the electrolyte solution is enabled to include the lithium salt that meets the foregoing relational expression, so that a sufficient lithium source may be provided for the battery, thereby increasing a lithium ion migration rate to improve conductivity, and providing a guarantee for long-cycling performance of the battery.

In this embodiment of the present disclosure, the battery electrolyte solution is enabled to include an organic solvent, an additive, and an electrolyte salt. The organic solvent includes an ethyl group solvent, the additive includes fluoroethylene carbonate, and the electrolyte salt includes a lithium salt. Percentages of the ethyl group solvent, the fluoroethylene carbonate, and the lithium salt in a total mass of the electrolyte solution are configured as follows: 0.4−N³≤A+B²+C²≤5 0.2−N³. N denotes a peeling strength value of the negative electrode plate, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution. A relatively robust solid electrolyte interface (Solid Electrolyte Interface, SEI) film may be formed on a surface of the negative electrode plate while improving infiltration of the negative electrode plate. The SEI film covering the surface of the negative electrode plate inhibits a side reaction between a negative electrode active material and the electrolyte solution, so as to reduce accumulation of a side reaction product. In this way, a peeling strength of the negative electrode plate may be increased, which further reduces a negative electrode impedance (also reduces an internal resistance of the battery), thereby reducing a risk of self-heating and self-ignition of the battery, and improving safety performance of the battery.

In addition, the electrolyte solution is enabled to include the lithium salt that meets the foregoing relational expression, so that a sufficient lithium source may be provided for the battery, thereby increasing a lithium ion migration rate to improve conductivity, and providing a guarantee for low-temperature performance and long-cycling performance of the battery. In other words, the battery electrolyte solution provided in the embodiments of the present disclosure resolves a problem of how to improve safety performance of the battery while avoiding degradation of other performance of the battery.

Optionally, the value of the peeling strength N of the negative electrode plate ranges from 0.1 gf/mm to 2 gf/mm.

Optionally, the mass of the ethyl group solvent accounts for 40 wt % to 85 wt % of the total mass of the electrolyte solution.

Optionally, the mass of the fluoroethylene carbonate accounts for 5 wt % to 18 wt % of the total mass of the electrolyte solution.

Optionally, the mass of the lithium salt accounts for 12 wt % to 18 wt % of the total mass of the electrolyte solution.

During specific implementation, the peeling strength N of the negative electrode plate may be 0.1 gf/mm, 0.2 gf/mm, 0.3 gf/mm, 0.4 gf/mm, 0.5 gf/mm, 0.6 gf/mm, 0.7 gf/mm, 0.8 gf/mm, 0.9 gf/mm, 1.0 gf/mm, 1.1 gf/mm, 1.2 gf/mm, 1.3 gf/mm, 1.4 gf/mm, 1.5 gf/mm, 1.6 gf/mm, 2.0 gf/mm, or the like, or may be any value within a range composed of any two of these values. When the value of the peeling strength N of the negative electrode plate ranges from 0.1 gf/mm to 2 gf/mm, a better synergistic effect may be achieved between the negative electrode plate and the electrolyte solution, thereby further improving safety performance of the battery while avoiding degradation of other performance of the battery.

The mass of the ethyl group solvent may account for 40 wt %, 50 wt %, 54 wt %, 60 wt %, 68 wt %, 70 wt %, 71 wt %, 80 wt %, 85 wt %, or the like of the total mass of the electrolyte solution, or may be any value within a range composed of any two of these values. When the mass of the ethyl group solvent accounts for 40 wt % to 85 wt % of the total mass of the electrolyte solution, a better synergistic effect may be achieved between the negative electrode plate and the electrolyte solution, thereby further improving safety performance of the battery while avoiding degradation of other performance of the battery.

The mass of the fluoroethylene carbonate may account for 5 wt %, 7 wt %, 9 wt %, 10 wt %, 12 wt %, 13 wt %, 14 wt %, 16 wt %, 18 wt % or the like of the total mass of the electrolyte solution, or may be any value within a range composed of any two of these values. When the mass of the fluoroethylene carbonate accounts for 5 wt % to 18 wt % of the total mass of the electrolyte solution, a better synergistic effect may be achieved between the negative electrode plate and the electrolyte solution, thereby further improving safety performance of the battery while avoiding degradation of other performance of the battery.

The mass of the lithium salt may account for 12 wt %, 13 wt %, 14 wt %, 15 wt %, 16 wt %, 17 wt %, 18 wt %, or the like of the total mass of the electrolyte solution, or may be any value within a range composed of any two of these values. When the mass of the lithium salt accounts for 12 wt % to 18 wt % of the total mass of the electrolyte solution, a better synergistic effect may be achieved between the negative electrode plate and the electrolyte solution, thereby further improving safety performance of the battery while avoiding degradation of other performance of the battery.

When the peeling strength of the negative electrode plate, the mass of the ethyl group solvent, the mass of the fluoroethylene carbonate, and the mass of the lithium salt all meet the foregoing value range, a better synergistic effect may be achieved between the negative electrode plate and the electrolyte solution, compared to a case in which one/two/three of the peeling strength of the negative electrode plate, the mass of the ethyl group solvent, the mass of the fluoroethylene carbonate, and the mass of the lithium salt meets (one of them meets, two of them meets, or three of them meets) the foregoing value range.

Optionally, the lithium salt includes at least one of lithium bis(trifluoromethanesulphonyl)imide, lithium bis(fluorosulfonyl)imide, lithium hexafluorophosphate, lithium difluorophosphate, lithium bisoxalate borate, lithium difluoro(oxalato)borate, lithium difluoro oxalate phosphate, lithium tetrafluoroborate, or lithium tetrafluoro(oxalato)phosphate.

Optionally, the lithium salt includes at least one of lithium bis(trifluoromethanesulphonyl)imide, lithium bis(fluorosulfonyl)imide, or lithium hexafluorophosphate. For example, the lithium salt may be lithium bis(trifluoromethanesulphonyl)imide, or a mixture of lithium bis(fluorosulfonyl)imide and lithium hexafluorophosphate.

Optionally, the electrolyte solution further includes a thiophene compound, and a structural formula of the thiophene compound is as follows:

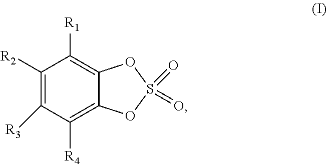

where R₁ is any one of hydrogen, halogen, and an alkyl carbon chain, R₂ is any one of hydrogen, halogen, and an alkyl carbon chain, R₃ is any one of hydrogen, halogen, and an alkyl carbon chain, R₄ is any one of hydrogen, halogen, and an alkyl carbon chain, and a quantity of carbon atoms in an alkyl carbon chain ranges from 1 to 10.

During specific implementation, R₁, R₂, R₃, and R₄ may be completely identical, may be partially identical, or may be completely different from one another. A quantity of carbon atoms in the alkyl carbon chain may be 1, 2, 3, 4, 5, 6, 7, 8, 9, or 10. The thiophene compound may be used as the additive in the electrolyte solution.

Enabling the electrolyte solution to include the thiophene compound may cause the thiophene compound to undergo polymerization reaction on the surfaces of both the positive electrode plate and the negative electrode plate, so as to form a mesh-like passivation film. The formed passivation film has a relatively small impedance, and may cover the surface of the positive electrode active material, thereby effectively preventing a side reaction, on the surface of the positive electrode plate, caused by oxygen release from the positive electrode active material. In addition, the mesh-like passivation film may further cover the surface of the negative electrode active material, so as to inhibit a side reaction of the negative electrode active material on the surface of the negative electrode plate. Since the side reaction may produce a side reaction product that increases an impedance, preventing a side reaction of the positive electrode active material can reduce an increase in an impedance of the battery during a cycling process, thereby improving cycling performance of the battery, achieving a better synergistic effect between the negative electrode plate and the electrolyte solution, and further improving low-temperature performance, high-temperature performance, and safety performance of the battery.

Optionally, the halogen is any one of fluorine, chlorine, and bromine. For example, R₁ is fluorine, R₂ is bromine, R₃ is hydrogen, and R₄ is the alkyl carbon chain.

Optionally, at least one carbon or hydrogen in the alkyl carbon chain is substituted by oxygen or halogen.

During specific implementation, at least one carbon in the alkyl carbon chain may be substituted by oxygen or halogen, or at least one hydrogen in the alkyl carbon chain may be substituted by oxygen or halogen, or at least one carbon and at least one hydrogen in the alkyl carbon chain may be substituted by oxygen or halogen.

Optionally, a structural formula of the thiophene compound is any one of following:

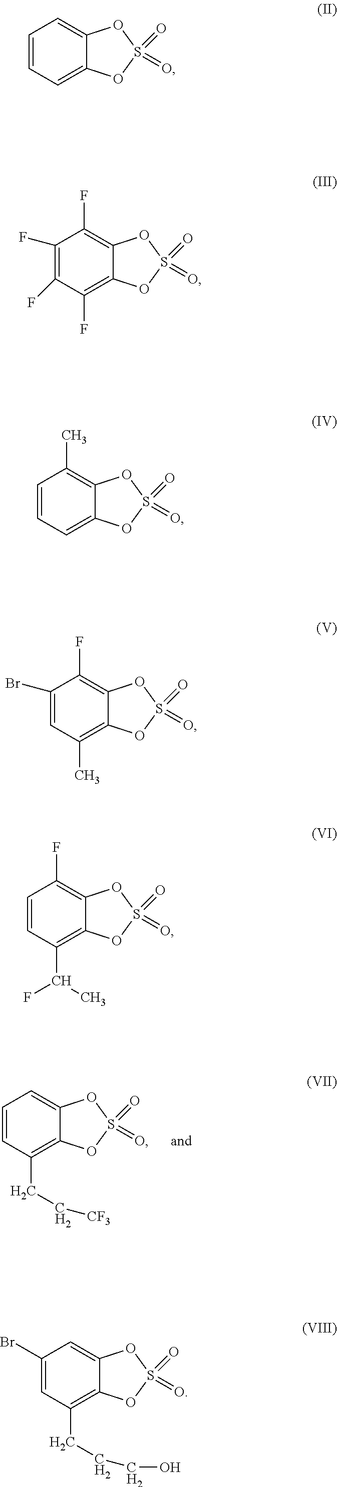

When the structural formula of the thiophene compound is any one of the foregoing structural formulas, the thiophene compound may form a denser and lower-impedance mesh-like passivation film on the surfaces of the positive and negative electrode plates. Therefore, cycling performance of the battery is further improved, so that a better synergistic effect is achieved between the negative electrode plate and the electrolyte solution, thereby further improving low-temperature performance, high-temperature performance, and safety performance of the battery.

Optionally, a mass of the thiophene compound accounts for 0.1 wt % to 2 wt % of the total mass of the electrolyte solution. During specific implementation, the mass of the thiophene compound accounts for 0.1 wt %, 0.5 wt %, 0.6 wt %, 0.9 wt %, 1.1 wt %, 1.6 wt %, 1.7 wt %, 1.8 wt %, 2 wt %, or the like of the total mass of the electrolyte solution, or may be any value within a range composed of any two of these values. When the mass of the thiophene compound accounts for 0.1 wt % to 2 wt % of the total mass of the electrolyte solution, a better synergistic effect may be achieved between the negative electrode plate and the electrolyte solution, thereby further improving safety performance of the battery while avoiding degradation of other performance of the battery.

Optionally, the additive may further include another additive different from the fluoroethylene carbonate, and the another additive includes one or more of a nitrile compound, a sulfur-containing compound, and a carbonate compound.

Optionally, the nitrile compound is selected from one or more of succinonitrile, glutaronitrile, adiponitrile, pimelonitrile, suberonitrile, glycerol trinitrile, ethoxy(pentafluoro)phosphazene, or 1,3,6-hexanetricarbonitrile.

Optionally, the sulfur-containing compound is selected from one or more of 1,3-propane sultone, 1-propene 1,3-sultone, ethylene sulfate, or vinylene sulfate.

Optionally, the carbonate compound is one or both of ethylene carbonate or vinyl ethylene carbonate.

Optionally, a total mass of the another additive accounts for 0 wt % to 10 wt % of a total mass of a non-aqueous electrolyte solution. During specific implementation, the total mass of the another additive accounts for 0 wt %, 0.1 wt %, 1 wt %, 2 wt %, 3 wt %, 4 wt %, 5 wt %, 6 wt %, 7 wt %, 8 wt %, 9 wt %, 10 wt %, or the like, or may be any value within a range composed of any two of these values.

Optionally, the organic solvent further includes at least one of carbonate, carboxylic acid ester, or fluorinated ether. Optionally, the carbonate includes at least one of ethylene carbonate, propylene carbonate, dimethyl carbonate, ethyl methyl carbonate, diethyl carbonate, or methyl propyl carbonate. The carboxylic acid ester includes at least one of ethyl propionate or propyl propionate. The fluorinated ether may be 1,1,2,3-tetrafluoroethyl-2,2,3,3-tetrafluoropropyl ether.

An embodiment of the present disclosure further provides a preparation method of a battery electrolyte solution. The method is used for preparing the battery electrolyte solution provided in embodiments of the present disclosure. A percentage of each of the ethyl group solvent, the fluoroethylene carbonate, and the lithium salt in the battery electrolyte solution in the total mass of the electrolyte solution is determined based on a peeling strength expected to be reached after the negative electrode plate is infiltrated with the electrolyte solution, such that the percentages of the ethyl group solvent, the fluoroethylene carbonate, and the lithium salt in the total mass of the electrolyte solution meet:

0.4−N³≤A+B²+C²≤5.2−N³,

where N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

In the embodiments of the present disclosure, the percentage of each of the ethyl group solvent, the fluoroethylene carbonate, and the lithium salt in the battery electrolyte solution in the total mass of the electrolyte solution is determined based on a peeling strength expected to be reached after the negative electrode plate is infiltrated with the electrolyte solution, such that the percentages of the ethyl group solvent, the fluoroethylene carbonate, and the lithium salt in the total mass of the electrolyte solution meet: 0.4−N³≤A+B²+C²≤5.2−N³. N denotes a peeling strength value of the negative electrode plate, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution. A relatively robust SEI film may be formed on a surface of the negative electrode plate while improving infiltration of the negative electrode plate. The SEI film covering the surface of the negative electrode plate inhibits a side reaction between a negative electrode active material and the electrolyte solution, so as to reduce accumulation of a side reaction product. In this way, a peeling strength of the negative electrode plate may be increased, which further reduces a negative electrode impedance (also reduces an internal resistance of the battery), thereby reducing a risk of self-heating and self-ignition of the battery, and improving safety performance of the battery.

In addition, the electrolyte solution is enabled to include the lithium salt that meets the foregoing relational expression, so that a sufficient lithium source may be provided for the battery, thereby increasing a lithium ion migration rate to improve conductivity, and providing a guarantee for low-temperature performance and long-cycling performance of the battery. In other words, the battery electrolyte solution provided in the embodiments of the present disclosure resolves a problem of how to improve safety performance of the battery while avoiding degradation of other performance of the battery.

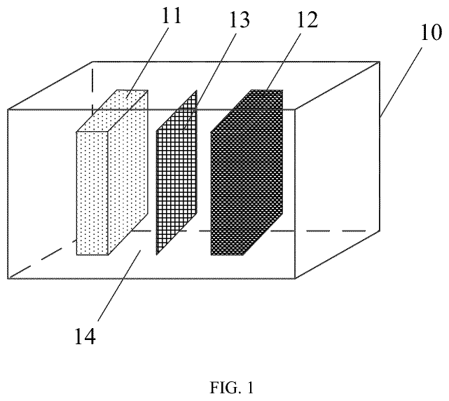

Referring to FIG. 1, an embodiment of the present disclosure further provides a battery 10. The battery includes a positive electrode plate 12, a negative electrode plate 11, and the electrolyte solution 14 provided in the embodiments of the present disclosure, both the positive electrode plate 12 and the negative electrode plate 11 are infiltrated with the electrolyte solution 14, and the battery 10 meets the following expression:

0.4−N³≤A+B²+C²≤5.2−N³,

where N denotes a peeling strength of the negative electrode plate 11 that is obtained by disassembling the battery 10, A denotes a percentage of a mass of the ethyl group solvent in the total mass of the electrolyte solution 14, B denotes a percentage of a mass of the fluoroethylene carbonate in the total mass of the electrolyte solution 14, and C denotes a percentage of a mass of the lithium salt in the total mass of the electrolyte solution 14.

It should be understood that a peeling strength value expected to be reached after the negative electrode plate 11 is infiltrated with the electrolyte solution 14 is generally equal to a peeling strength (that is, the peeling strength of the negative electrode plate 11 that is obtained by dismantling the battery 10) after the negative electrode plate 11 is infiltrated with the electrolyte solution 14.

During specific implementation, the battery 10 may be a wound battery, or may be a stacked battery, and the battery 10 further includes a separator 13 disposed between the positive electrode plate 12 and the negative electrode plate 11. The negative electrode plate 11, the separator 13, and the positive electrode plate 12 are sequentially stacked, and the positive electrode plate 12, the negative electrode plate 11, and the separator 13 are all infiltrated with the electrolyte solution 14. When the battery 10 is a stacked battery, a structure of the battery is shown in FIG. 1.

The positive electrode plate 12 includes a positive electrode current collector, and a positive electrode active material layer is applied on one or both sides of the positive electrode current collector. The positive electrode active material layer includes a positive electrode active material, a conductive agent, and a binder.

The positive electrode active material is selected from lithium cobaltate or lithium cobaltate doped and coated with two or more elements in Al, Mg, Mn, Cr, Ti, or Zr. A chemical formula of the lithium cobaltate doped and coated with two or more elements in Al, Mg, Mn, Cr, Ti, or Zr is LiₓGo_{1-y1-y2-y3-y4}E_{y1}F_{y2}G_{y3}Dy₄O₂, where 0.95≤x≤1.05, 0.01≤y1≤0.1, 0.01≤y2≤0.1, 0≤y3≤0.1, 0≤y4≤0.1, and E, F, G, and D are selected from two or more elements in Al, Mg, Mn, Cr, Ti, or Zr.

The negative electrode plate 11 includes a negative electrode current collector, and a negative electrode active material layer is applied on one or both sides of the negative electrode current collector. The negative electrode active material layer includes a negative electrode active material, a conductive agent, and a binder.

Optionally, the negative electrode active material is graphite.

Optionally, the negative electrode active material includes graphite, and the negative electrode active material further includes at least one of SiOₓ or Si, where 0<x<2.

A charge cut-off voltage of the battery provided in this embodiment of the present disclosure is 4.48 V or above.

For a structure and a working principle of the electrolyte solution provided in this embodiment of the present disclosure, reference may be made to the foregoing embodiment, and details are not described herein again. The battery provided in this embodiment of the present disclosure includes the electrolyte solution provided in the embodiments of the present disclosure. Therefore, the battery provided in this embodiment of the present disclosure has all beneficial effects of the electrolyte solution provided in the embodiments of the present disclosure.

In the battery provided in this embodiment of the present disclosure, when a total percentage of the ethyl group solvent in the electrolyte solution, a content of the fluoroethylene carbonate, a content of the lithium salt, and the peeling strength of the negative electrode plate meet the foregoing relational expression, a better synergistic effect is achieved between the negative electrode plate and the electrolyte solution. Specifically, a relatively robust SEI film may be formed on a surface of the negative electrode plate while improving infiltration of the negative electrode plate. The SEI film reduces a side reaction between the negative electrode active material and the electrolyte solution, so as to reduce accumulation of a side reaction product. In this way, a peeling strength of the negative electrode plate is increased, which further reduces an internal resistance of the battery, thereby reducing a risk of self-heating and self-ignition of the battery, improving safety performance of the battery, and improving other performance of the battery. In addition, the electrolyte solution is enabled to include the lithium salt that meets the foregoing relational expression, thereby increasing a lithium ion migration rate to improve conductivity, and providing a guarantee for low-temperature performance and long-cycling performance of the battery.

The following describes, with specific experiments, the battery provided in this embodiment of the present disclosure.

Lithium-ion batteries in Comparative Examples 1 to 5 and Examples 1 to 10 were prepared according to the following preparation method, and a difference lies only in peeling strengths of negative electrode plates and electrolyte solutions. The difference is specifically shown in Table 1.

### (1) Preparation of a Positive Electrode Plate

A positive electrode active material LiCoO₂ (having a specific surface area shown in Table 1), a binder polyvinylidene fluoride (PVDF), and a conductive agent acetylene black were mixed at a weight ratio of 98.2:1.1:0.7, and were added with N-methylpyrrolidone (NMP). The mixture was stirred under action of a vacuum mixer until a mixed system became a positive electrode slurry with uniform fluidity. The positive electrode slurry was evenly applied on aluminum foil having a thickness of 12 m. The coated aluminum foil was baked in a five-stage oven with different temperatures and then dried in an oven at 120° C. for eight hours, followed by rolling and cutting, to obtain required different positive electrode plates.

### (2) Preparation of a Negative Electrode Plate

A negative electrode active material artificial graphite with a mass percentage of 96.5%, a conductive agent single-walled carbon nanotube (SWCNT) with a mass percentage of 0.2%, a conductive agent conductive carbon black (SP) with a mass percentage of 0.8%, a binder sodium carboxymethyl cellulose (CMC) with a mass percentage of 1.2%, and a binder styrene-butadiene rubber (SBR) with a mass percentage of 1.3% were made into a slurry by using a wet process. The slurry was applied on a surface of a negative electrode current collector made of copper foil, and then drying (temperature: 85° C., time: 5 h), rolling, and die cutting were carried out to obtain required negative electrode plates with different peeling strengths (the peeling strengths are specifically shown in Table 1).

### (3) Preparation of an Electrolyte Solution

In a glove box filled with argon (moisture <10 ppm, oxygen <1 ppm), ethylene carbonate (EC) and propylene carbonate (PC) were evenly mixed at a mass ratio of 2:1, an ethyl group solvent accounting for 40 wt % to 85 wt % of a total mass of a non-aqueous electrolyte solution (specific percentages of the ethyl group solvent are shown in Table 1), and LiPF₆ accounting for 12 wt % to 18 wt % of the total mass of the non-aqueous electrolyte solution (specific percentages of the LiPF₆ are shown in Table 1), and an additive (specific percentages and types of additives are shown in Table 1) were slowly added into the mixed solution. The mixture was stirred evenly to obtain the non-aqueous electrolyte solution.

### (4) Preparation of a Separator

A polyethylene separator with a thickness ranging from 7 μm to 9 μm is selected.

### (5) Preparation of a Lithium-Ion Battery

The foregoing prepared positive electrode plate, separator, and negative electrode plate were wound to obtain an unfilled bare cell. The bare cell was placed in outer packaging foil, the prepared electrolyte solution was injected into the dried bare cell, and after processes such as vacuum packaging, standing, formation, shaping, and sorting, the lithium-ion battery required was obtained.

TABLE 1

| | A percentage | | A | | | | |
|---|---|---|---|---|---|---|---|
| | of a mass of | A percentage | percentage | Peeling | | Thiophene | |
| | the ethyl | of a mass of | of a mass | strength | | compound | |
| | group | fluoroethylene | of Lithium | N | N³ + A + | | Content/ |
| Item | solvent A | carbonate B | salt C | (gf/mm) | B² + C² | Type | wt % |
| Comparative | 20% | 10% | 14% | 0.8 | 0.74 | / | / |
| Example 1 | | | | | | | |
| Comparative | 50% | 0% | 14% | 0.8 | 1.03 | / | / |
| Example 2 | | | | | | | |
| Comparative | 50% | 10% | 5.0% | 0.8 | 1.02 | / | / |
| Example 3 | | | | | | | |
| Comparative | 50% | 10% | 14% | 0.05 | 0.53 | / | / |
| Example 4 | | | | | | | |
| Comparative | 50% | 10% | 14% | 1.8 | 6.36 | / | / |
| Example 5 | | | | | | | |
| Example 1 | 50% | 10% | 14% | 0.8 | 1.04 | / | / |
| Example 2 | 40% | 5% | 13% | 0.5 | 0.54 | / | / |
| Example 3 | 55% | 7% | 12% | 0.6 | 0.79 | / | / |
| Example 4 | 60% | 8% | 15% | 1 | 1.63 | / | / |
| Example 5 | 45% | 9% | 14.5% | 1.2 | 2.21 | II | 0.3% |
| Example 6 | 85% | 15% | 16% | 1.6 | 4.99 | III | 2% |
| Example 7 | 70% | 12% | 18% | 0.4 | 0.81 | IV | 0.5% |
| Example 8 | 75% | 18% | 15.5% | 0.3 | 0.83 | II | 0.1% |
| Example 9 | 50% | 10% | 14% | 0.8 | 1.04 | II | 0.2% |

A structural formula of the thiophene compound is any one of following:

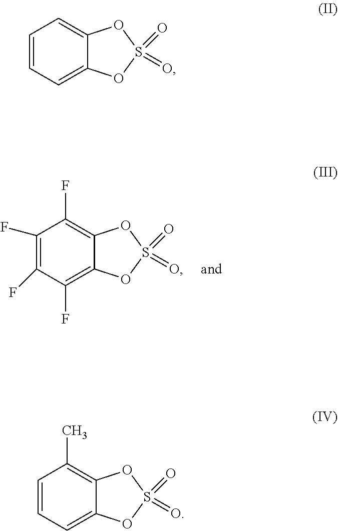

Example 10: Reference is made to Example 1 for implementation, with a difference being that the lithium salt in this example is, instead of lithium hexafluorophosphate, a mixture of equal amounts of lithium bis(fluorosulfonyl)imide and lithium hexafluorophosphate, with a mass ratio of the two lithium salts of 1:1.

The batteries in Comparative Examples 1 to 5 and Examples 1 to 10 were tested for electrochemical performance. The related descriptions are as follows:

Peeling force strength test: Each of the negative electrode plates in Comparative Examples 1 to 5 and Examples 1 to 10 is cut into a sample strip of 24 mm×15 cm, covered with a glass slide, pressed back and forth by using a roller, and then tested with a tensile machine at a speed of 200 mm/min, to obtain a testing result that is a peel force of P (in a unit of gf).

A calculation formula used is as follows: Peeling strength N (gf/mm)=P/24 mm (width).

(1) 10° C. cycling test: The batteries in Comparative Examples 1 to 5 and Examples 1 to 10 were placed in an environment of (10±2)° C. to stand for two to three hours. When the battery bodies reached (10±2)° C., the batteries were charged at a constant current of 0.7C, with a cut-off current of 0.05C. After the batteries were fully charged, the batteries were left aside for five minutes, and then discharged at a constant current of 0.5C to a cut-off voltage of 3.0 V. A highest discharge capacity for the first three cycles was recorded as an initial capacity Q. When the number of cycles reaches 300, the last discharge capacity of the battery was recorded as Q1. Recorded results are shown in Table 2.

A calculation formula used is as follows: Capacity retention rate (%)=Q1/Q×100%.

(2) 70° C. and 48-hour high-temperature storage test: The batteries obtained in the Examples and Comparative Examples were charged and discharged three times at a charge and discharge rate of 0.5C at room temperature, and then charged to a fully charged state at a rate of 0.5C. A highest discharge capacity Q2 of the first three cycles at 0.5C was recorded. The batteries in a fully charged state were stored at 70° C. for 48 hours. After 48 hours, a 0.5C discharge capacity Q3 for each battery was recorded. Then, experimental data such as a capacity retention rate and whether gas is generated that are stored at a high temperature of each battery were obtained by calculation. Recorded results are shown in Table 2.

A calculation formula used is as follows:

Capacity retention rate (%)=Q3/Q2×100%

(3) In a nail penetration test, a high-temperature resistant steel needle (a cone angle of the needle tip ranges from 45° to 60°, and a surface of the needle is smooth without rust, oxide layer, or grease) with a diameter of ϕ ranging from 5 mm to 8 mm penetrated through the batteries in Examples 1 to 9 and Comparative Examples 1 to 10 from a direction perpendicular to electrode plates of the batteries at a speed of (25±5) mm/s, and a penetration position was preferably close to a geometric center of a penetrated surface (the steel needle remained in the batteries). Based on observation, after one hour or when a maximum temperature of surfaces of the batteries dropped to a peak temperature of 10° C. or below, the test ended.

(4) Low-temperature discharge test: The batteries in Comparative Examples 1 to 5 and Examples 1 to 10 were first discharged at 0.2C to 3.0 V at an ambient temperature of (25±3)° C., and left aside for five minutes. The batteries were charged at 0.7C, and when a voltage across cell terminals reached a charging limit voltage, the batteries began to be charged at a constant voltage. The charging was not stopped until a charging current is less than or equal to a cut-off current. The batteries were left aside for five minutes and then discharged at 0.2C to 3.0 V, and a discharge capacity in this case was recorded as a normal-temperature capacity Q4. Then, the cells were charged at 0.7C, and when a voltage across the cell terminals reached a charging limit voltage, the batteries began to be charged at a constant voltage. The charging was not stopped until a charging current is less than or equal to a cut-off current. The fully charged batteries were left aside at (−20±2)° C. for four hours, and then discharged at a current of 0.2C to a cut-off voltage of 3.0 V. A discharge capacity Q5 was recorded to calculate a low-temperature discharge capacity retention rate. Recorded results are shown in Table 2.

A calculation formula used is as follows: Low-temperature discharge capacity retention rate (%)=Q5/Q4×100%.

(5) 130° C. thermal shock test: The batteries in Comparative Examples 1 to 5 and Examples 1 to 10 were heated in a convection mode or by using a circulation hot air box at a start temperature of (25±3)° C., with a temperature change rate of (5±2)° C./min. The temperature was raised to (130±2)° C., the batteries were kept in the temperature for 60 minutes, and state results of the batteries were recorded and are shown in Table 2.

TABLE 2

| | | | | | Safety test (Criteria for passing | |
|---|---|---|---|---|---|---|
| | | | | | this test are that neither fire nor | |
| | Capacity | 70° C. | | Capacity | explosion occurs) | |
| | retention | high-temperature | | retention | Nail | 130° C. |
| | rate after | storage for 48 hours | | rate after | penetration | thermal shock |
| | 300 cycles | Capacity | | discharge | (number of | (number of |
| | at 10° C. | retention | Battery | at −20° C. | passed/number | passed/number |
| Group | and 0.7 C | rate (%) | state | and 0.2 C | of tested) | of tested) |
| Comparative | 42.1% | 60.7% | Gassing | 33.1% | 1/5 | 0/5 |
| Example 1 | | | | | | |
| Comparative | 21.0% | 43.1% | Gassing | 25.0% | 2/5 | 1/5 |
| Example 2 | | | | | | |
| Comparative | 27.4% | 62.5% | Gassing | 22.4% | 0/5 | 0/5 |
| Example 3 | | | | | | |
| Comparative | 38.8% | 51.4% | Gassing | 32.8% | 1/5 | 1/5 |
| Example 4 | | | | | | |
| Comparative | 32.8% | 58.8% | Gassing | 35.8% | 0/5 | 0/5 |
| Example 5 | | | | | | |
| Example 1 | 60.1% | 76.1% | No gassing | 41.1% | 5/5 | 5/5 |
| Example 2 | 65.3% | 78.4% | No gassing | 43.3% | 5/5 | 5/5 |
| Example 3 | 64.4% | 71.4% | No gassing | 47.4% | 5/5 | 5/5 |
| Example 4 | 62.1% | 75.0% | No gassing | 44.1% | 5/5 | 5/5 |
| Example 5 | 68.0% | 80.4% | No gassing | 49.0% | 5/5 | 5/5 |
| Example 6 | 71.3% | 82.3% | No gassing | 48.3% | 5/5 | 5/5 |
| Example 7 | 71.2% | 81.1% | No gassing | 42.0% | 5/5 | 5/5 |
| Example 8 | 74.5% | 85.4% | No gassing | 45.0% | 5/5 | 5/5 |
| Example 9 | 69.1% | 81.7% | No gassing | 46.8% | 5/5 | 5/5 |
| Example 10 | 62.3% | 77.3% | No gassing | 42.4% | 5/5 | 5/5 |

It may be learned from the experimental test results of the batteries in Comparative Examples 1 to 5 and Examples 1 to 10 in Table 2 that, through the synergistic effect between the negative electrode plate and the electrolyte solution of the lithium-ion battery, a side reaction between the negative electrode active material and the electrolyte solution can be inhibited when a value of N³+A+B²+C² ranges from 0.4 to 5.2, thereby reducing accumulation of a side reaction product, further increasing a peeling strength of the negative electrode plate, improving interfacial compatibility of the battery, and effectively improving low-temperature performance, high-temperature performance, and safety performance of the battery.

The embodiments of the present disclosure are described above with reference to the accompanying drawings, but the present disclosure is not limited to the foregoing specific implementations. The foregoing specific implementations are merely illustrative and nonrestrictive. Under the guidance of the present disclosure, a person of ordinary skill in the art can also make many forms without departing from the scope of protection of the present disclosure and the claims, all of which are within the protection of the present disclosure.

What is claimed is:

1. A battery electrolyte solution, comprising an organic solvent, an additive, and an electrolyte salt, wherein the organic solvent comprises an ethyl group solvent, the additive comprises fluoroethylene carbonate, the electrolyte salt comprises a lithium salt, and the electrolyte solution is in contact with a negative electrode plate; and
percentages of the ethyl group solvent, the fluoroethylene carbonate, and the lithium salt in a total mass of the electrolyte solution are configured as follows:
0.4−N³≤A+B²+C²≤5.2−N³,
wherein N denotes a peeling strength value of the negative electrode plate, in a unit of gf/mm, A denotes a percentage of a mass of the ethyl group solvent in the total mass of the electrolyte solution, B denotes a percentage of a mass of the fluoroethylene carbonate in the total mass of the electrolyte solution, and C denotes a percentage of a mass of the lithium salt in the total mass of the electrolyte solution.

2. The battery electrolyte solution according to claim 1, wherein the value of the peeling strength N of the negative electrode plate ranges from 0.1 gf/mm to 2 gf/mm.

3. The battery electrolyte solution according to claim 1, wherein the mass of the ethyl group solvent accounts for 40 wt % to 85 wt % of the total mass of the electrolyte solution.

4. The battery electrolyte solution according to claim 1, wherein the mass of the fluoroethylene carbonate accounts for 5 wt % to 18 wt % of the total mass of the electrolyte solution.

5. The battery electrolyte solution according to claim 1, wherein the mass of the lithium salt accounts for 12 wt % to 18 wt % of the total mass of the electrolyte solution.

6. The battery electrolyte solution according to claim 1, wherein the lithium salt comprises at least one of lithium bis(trifluoromethanesulphonyl)imide, lithium bis(fluorosulfonyl)imide, lithium hexafluorophosphate, lithium difluorophosphate, lithium bisoxalate borate, lithium difluoro(oxalato)borate, lithium difluoro oxalate phosphate, lithium tetrafluoroborate, or lithium tetrafluoro(oxalato)phosphate.

7. The battery electrolyte solution according to claim 6, wherein the lithium salt comprises at least one of lithium bis(trifluoromethanesulphonyl)imide, lithium bis(fluorosulfonyl)imide, or lithium hexafluorophosphate.

8. The battery electrolyte solution according to claim 1, wherein the electrolyte solution further comprises a thiophene compound, and a structural formula of the thiophene compound is as follows: 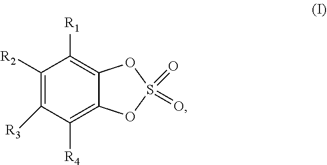
wherein R₁ is any one of hydrogen, halogen, and an alkyl carbon chain, R₂ is any one of hydrogen, halogen, and an alkyl carbon chain, R₃ is any one of hydrogen, halogen, and an alkyl carbon chain, R₄ is any one of hydrogen, halogen, and an alkyl carbon chain, and a quantity of carbon atoms in the alkyl carbon chain ranges from 1 to 10.

9. The battery electrolyte solution according to claim 8, wherein the halogen is any one of fluorine, chlorine, and bromine.

10. The battery electrolyte solution according to claim 8, wherein at least one carbon or hydrogen in the alkyl carbon chain is substituted by oxygen or halogen.

11. The battery electrolyte solution according to claim 8, wherein a structural formula of the thiophene compound is any one of following:

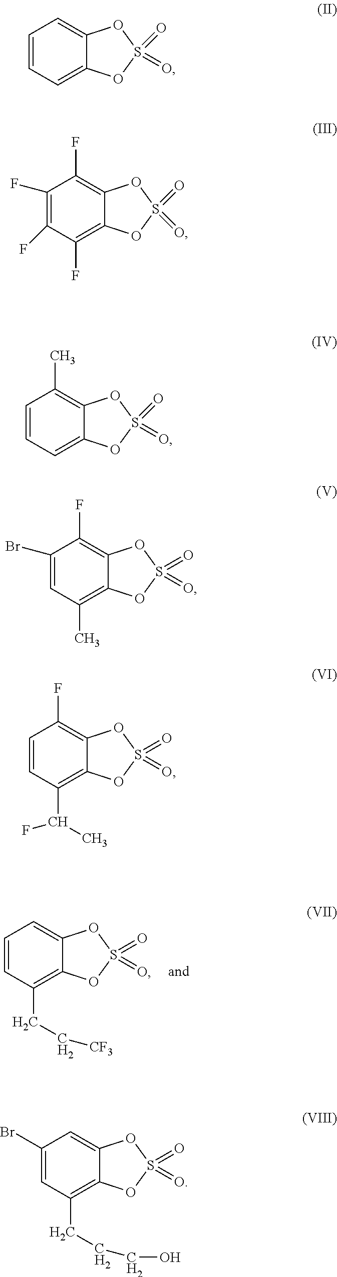

12. The battery electrolyte solution according to claim 8, wherein a mass of the thiophene compound accounts for 0.1 wt % to 2 wt % of the total mass of the electrolyte solution.

13. The battery electrolyte solution according to claim 1, wherein the additive further comprises one or more of a nitrile compound, a sulfur-containing compound, and a carbonate compound.

14. The battery electrolyte solution according to claim 13, wherein a total mass of the nitrile compound, the sulfur-containing compound, and the carbonate compound accounts for 0 wt % to 10 wt % of the total mass of the electrolyte solution.

15. The battery electrolyte solution according to claim 13, wherein the nitrile compound is selected from one or more of succinonitrile, glutaronitrile, adiponitrile, pimelonitrile, suberonitrile, glycerol trinitrile, ethoxy(pentafluoro)phosphazene, and 1,3,6-hexanetricarbonitrile.

16. The battery electrolyte solution according to claim 13, wherein the sulfur-containing compound is selected from one or more of 1-propene 1,3-sultone, ethylene sulfate, and vinylene sulfate.

17. The battery electrolyte solution according to claim 13, wherein the carbonate compound is one or more of ethylene carbonate, fluoroethylene carbonate, and vinyl ethylene carbonate.

18. The battery electrolyte solution according to claim 1, wherein the organic solvent further comprises at least one of carbonate, carboxylic acid ester, or fluorinated ether.

19. The battery electrolyte solution according to claim 18, wherein the carbonate comprises at least one of ethylene carbonate, propylene carbonate, dimethyl carbonate, ethyl methyl carbonate, diethyl carbonate, or methyl propyl carbonate; and/or
the carboxylic acid ester comprises at least one of ethyl propionate or propyl propionate; and/or
the fluorinated ether comprises 1,1,2,3-tetrafluoroethyl-2,2,3,3-tetrafluoropropyl ether.

20. A battery, comprising a positive electrode plate, a negative electrode plate, and the battery electrolyte solution according to claim 1, wherein both the positive electrode plate and the negative electrode plate are infiltrated with the battery electrolyte solution, and the battery meets the following expression:
0.4−N³≤A+B²+C²≤5.2−N³,
wherein N denotes a peeling strength of the negative electrode plate, in a unit of gf/mm, A denotes a percentage of a mass of the ethyl group solvent in a total mass of the electrolyte solution, B denotes a percentage of a mass of the fluoroethylene carbonate in the total mass of the electrolyte solution, and C denotes a percentage of a mass of the lithium salt in the total mass of the electrolyte solution.
